(19) United States
(12) Patent Application Publication
(10) Pub. No.: US 2025/0002426 A1
(43) Pub. Date: Jan. 2, 2025

(54) Staged Alkylation for Producing Xylene Products

(71) Applicant: ExxonMobil Chemical Patents Inc., Baytown, TX (US)

(72) Inventors: Krystle J. Emanuele, Houston, TX (US); Xiaobo ZHENG, Houston, TX (US); Robert G. TINGER, Friendswood, TX (US); Ali A. KHEIR, Houston, TX (US); Hsu CHIANG, Houston, TX (US); Seth M. WASHBURN, Houston, TX (US)

(21) Appl. No.: 18/700,359

(22) PCT Filed: Sep. 30, 2022
(86) PCT No.: PCT/US2022/077328

§ 371 Date: Apr. 11, 2024

Related U.S. Application Data
(60) Provisional application No. 63/262,421, filed on Oct. 12, 2021.

(51) Int. Cl. C07C 2/86; B01J 8/04; B01J 29/70

(52) U.S. Cl. CPC C07C 2/864; B01J 8/0492; B01J 29/7038; C07C 2529/70

(57) ABSTRACT

Processes and systems for converting benzene and/or toluene via methylation with methanol and/or dimethyl ether may be performed by contacting an aromatic hydrocarbon feed with a first methylating agent feed in the presence of a methylation catalyst in a series of fixed beds. Between the fixed beds, the product mixture from the upstream bed may be treated by (a) reducing the temperature, (b) adding additional methylating agent feed, (c) optionally removing water, and (d) optionally adding additional aromatic hydrocarbon feed.

### CROSS-REFERENCE TO RELATED APPLICATION

This application claims the benefit of U.S. Provisional Patent Application 63/262,421 filed 12 Oct. 2022 entitled “STAGED ALKYLATION FOR PRODUCING XYLENE PRODUCTS,” the entirety of which is incorporated by reference herein.

### FIELD

This disclosure relates to processes and systems for converting benzene and/or toluene. In particular, this disclosure relates to processes and systems for converting benzene and/or toluene via methylation with methanol and/or dimethyl ether. This disclosure is useful, for example, in making p-xylene and/or o-xylene via benzene/toluene methylation with methanol and/or dimethyl ether.

### BACKGROUND

1,4-Dimethylbenzene (para-xylene, or p-xylene) is a valuable chemical feedstock and is used mainly for the production of terephthalic acid and polyethylene terephthalate resins, in order to provide synthetic textiles, bottles, and plastic materials among other industrial applications. As commercial applications of p-xylene have increased, there has been an increased need for more selective processes and increased yields for p-xylene production. Worldwide production capacity of p-xylene is about 40 million tons per year, and the continually increasing demand for purified terephthalic acid in polyester production processes is projected to provide a corresponding demand to the p-xylene market. Thus, there has been a corresponding increase in demand for the development of efficient and cost-effective p-xylene formation and isolation processes.

p-Xylene can be extracted from the BTX aromatics (benzene, toluene and xylene isomers) in the catalytic reformate produced by catalytic reforming of petroleum naphtha. Alternatively, p-xylene can be produced via toluene disproportionation, toluene transalkylation with C9+ aromatics, or toluene methylation with methanol. Regardless of the method of production, p-xylene is then separated out in a series of distillation, adsorption, crystallization and reaction processes from other C8 aromatic isomers, such as meta-xylene, ortho-xylene, and ethylbenzene. The melting point of p-xylene is the highest among such series of isomers, but simple crystallization does not allow easy purification due to the formation of eutectic mixtures. Consequently, current technologies for p-xylene production are energy intensive, and p-xylene separation and purification are a major cost factor in the production of p-xylene. Hence, alternative methods to selectively produce p-xylene are still needed.

The methylation of toluene or benzene is a favored route to the formation of p-xylene because of the low cost of starting materials and the potential to provide high yields. One methylation method uses methanol as an alkylation reagent and zeolites (or selectivated zeolites) as catalysts. However, the reaction temperatures and other process conditions cause rapid catalyst deactivation, significant light gas generation through methanol to olefin chemistry, and production of other trace by-products that have to be removed from the product. Systems and methods for improving the p-xylene production and increasing catalyst life would be of value in the art.

### SUMMARY

This disclosure relates to processes and systems for converting benzene and/or toluene. In particular, this disclosure relates to processes and systems for converting benzene and/or toluene via methylation with methanol and/or dimethyl ether. This disclosure is useful, for example, in making p-xylene and/or o-xylene via benzene/toluene methylation with methanol and/or dimethyl ether.

A non-limiting example process of the present disclosure for producing p-xylene may comprise at least one of: (I) contacting an aromatic hydrocarbon feed with a first methylating agent feed in the presence of a first methylation catalyst in a first fixed bed under a first set of methylation reaction conditions to produce a first methylation product mixture comprising p-xylene exiting the first fixed bed, wherein the aromatic hydrocarbon feed comprises benzene and/or toluene, wherein the first methylating agent feed comprises methanol and/or dimethyl ether, and wherein the first methylation product mixture when exiting the first fixed bed has a first temperature of T1; (II) producing a mixture feed having a second temperature of T2 via steps comprising: (IIa) reducing a temperature of the first methylation product mixture and (IIb) injecting a second methylating agent feed having a fourth temperature T4 into the first methylation product mixture, wherein step (IIa) occurs before, after, and/or during step (IIb), and wherein the second methylating agent feed comprises methanol and/or dimethyl ether; and (III) contacting the mixture feed with a second methylation catalyst in a second fixed bed under a second set of methylation reaction conditions to produce a second methylation product mixture comprising p-xylene exiting the second fixed bed, wherein the second methylation product mixture when exiting the second fixed bed has a third temperature of T3.

Another non-limiting example process of the present disclosure for producing p-xylene may comprise one or more of: (I) contacting an aromatic hydrocarbon feed with a first methylating agent feed in the presence of a first methylation catalyst in a first fixed bed under a first set of methylation reaction conditions to produce a first methylation product mixture comprising p-xylene exiting the first fixed bed, wherein the aromatic hydrocarbon feed comprises benzene and/or toluene, wherein the first methylating agent feed comprises methanol and/or dimethyl ether, and wherein the first methylation product mixture when exiting the first fixed bed has a first temperature of T1; (II) producing a mixture feed having a second temperature of T2 via steps comprising: (IIa) reducing a temperature of the first methylation product mixture, (IIb) injecting a second methylating agent feed having a fourth temperature T4 into the first methylation product mixture, and (IIc) removing at least a portion of water from the first methylation product mixture, wherein the second methylating agent feed comprises methanol and/or dimethyl ether, and step (IIa), step (IIb), and step (IIc) independently occur in any order and/or simultaneously; and (III) contacting the mixture feed with a second methylation catalyst in a second fixed bed under a second set of methylation reaction conditions to produce a second methylation product mixture comprising p-xylene exiting the second fixed bed, wherein the second methylation product mixture when exiting the second fixed bed has a third temperature of T3.

Another non-limiting example process of the present disclosure for producing p-xylene may comprise one or more of; (I) contacting an aromatic hydrocarbon feed with a first methylating agent feed in the presence of a first methylation catalyst in a first fixed bed under a first set of methylation reaction conditions to produce a first methylation product mixture comprising p-xylene exiting the first fixed bed, wherein the aromatic hydrocarbon feed comprises benzene and/or toluene, wherein the first methylating agent feed comprises methanol and/or dimethyl ether, and wherein the first methylation product mixture when exiting the first fixed bed has a first temperature of T1; (II) producing a mixture feed having a second temperature of T2 via steps comprising: (IIa) reducing a temperature of the first methylation product mixture, (IIb) injecting a second methylating agent feed having a fourth temperature T4 into the first methylation product mixture, and (IIc) injecting a second aromatic hydrocarbon feed having a fifth temperature T5 into the first methylation product mixture, wherein the second aromatic hydrocarbon feed comprises benzene and/or toluene, wherein the second methylating agent feed comprises methanol and/or dimethyl ether, and wherein step (IIa), step (IIb), and step (IIc) independently occur in any order and/or simultaneously; and (III) contacting the mixture feed with a second methylation catalyst in a second fixed bed under a second set of methylation reaction conditions to produce a second methylation product mixture comprising p-xylene exiting the second fixed bed, wherein the second methylation product mixture when exiting the second fixed bed has a third temperature of T3.

Another non-limiting example process of the present disclosure for producing p-xylene may comprise at least one of: (I) contacting an aromatic hydrocarbon feed with a first methylating agent feed in the presence of a first methylation catalyst in a first fixed bed under a first set of methylation reaction conditions to produce a first methylation product mixture comprising p-xylene exiting the first fixed bed, wherein the aromatic hydrocarbon feed comprises benzene and/or toluene, wherein the first methylating agent feed comprises methanol and/or dimethyl ether, and wherein the first methylation product mixture when exiting the first fixed bed has a first temperature of T1; (II) producing a mixture feed having a second temperature of T2 via steps comprising: (IIa) reducing a temperature of the first methylation product mixture, (IIb) injecting a second methylating agent feed having a fourth temperature T4 into the first methylation product mixture, (IIc) injecting a second aromatic hydrocarbon feed having a fifth temperature T5 into the first methylation product mixture, and (IId) removing at least a portion of water from the first methylation product mixture, wherein the second methylating agent feed comprises methanol and/or dimethyl ether, wherein the second aromatic hydrocarbon feed comprises benzene and/or toluene, and wherein step (IIa), step (IIb), step (IIc), and step (IId) independently occur in any order and/or simultaneously; and (III) contacting the mixture feed with a second methylation catalyst in a second fixed bed under a second set of methylation reaction conditions to produce a second methylation product mixture comprising p-xylene exiting the second fixed bed, wherein the second methylation product mixture when exiting the second fixed bed has a third temperature of T3.

These and other features and attributes of the disclosed processes of the present disclosure and their advantageous applications and/or uses will be apparent from the detailed description which follows.

### BRIEF DESCRIPTION OF THE DRAWINGS

FIG. 1 illustrates a flow diagram of a non-limiting example method of the present disclosure for converting benzene/toluene via methylation with methanol/dimethyl ether to produce xylenes comprising recycling of DME, methanol, and toluene to the methylation reactor, according to one embodiment of this disclosure.

FIG. 2 is a schematic diagram showing a process of this disclosure for converting benzene/toluene via methylation with methanol/dimethyl ether to produce xylenes comprising recycling of DME, methanol, and toluene to the methylation reactor, according to one embodiment of this disclosure.

FIG. 3 is a schematic diagram showing a process of this disclosure for converting benzene/toluene via methylation with methanol/dimethyl ether to produce xylenes comprising a separation subsystem including a plurality of separation units which allow for recycling of DME, methanol, and toluene to the methylation reactor, according to one embodiment of this disclosure.

FIG. 4 is a plot of p-xylene selectivity and overall xylene conversion simulated using a kinetic model for a method for converting benzene/toluene via methylation with methanol/dimethyl ether to produce xylenes comprising recycling of DME, methanol, and toluene to the methylation reactor, according to one embodiment of this disclosure.

FIG. 5 is a plot of temperature and p-xylene yield as a function of reactor length for a staged method, according to one embodiment of this disclosure.

### DETAILED DESCRIPTION

In this disclosure, a process is described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other step, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described.

Unless otherwise indicated, all numbers indicating quantities in this disclosure are to be understood as being modified by the term “about” in all instances. It should also be understood that the numerical values used in the specification and claims constitute specific embodiments. Efforts have been made to ensure the accuracy of the data in the examples. However, it should be understood that any measured data inherently contain a certain level of error due to the limitation of the technique and equipment used for making the measurement.

As used herein, the indefinite article “a” or “an” shall mean “at least one” unless specified to the contrary or the context clearly indicates otherwise. Thus, embodiments using “an ether” include embodiments where one, two or more ethers are used, unless specified to the contrary or the context clearly indicates that only one ether is used.

For the purposes of this disclosure, the nomenclature of elements is pursuant to the version of Periodic Table of Elements as described in CHEMICAL AND ENGINEERING NEWS, 63(5), pg. 27 (1985).

The following abbreviations may be used herein for the sake of brevity: RT is room temperature (and is 23° C. unless otherwise indicated), kPag is kilopascal gauge, psig is pound-force per square inch gauge, psia is pounds per square inch absolute, and WHSV is weight hourly space velocity. Abbreviations for atoms are as given in the periodic table (Li=lithium, for example).

The term “Cn” compound or group, wherein n is a positive integer, means a compound or a group comprising carbon atoms therein at the number of n. Thus, a “Cm to Cn” alkyl means an alkyl group comprising carbon atoms therein at a number in a range from m to n. Thus, a C1-C3 alkyl means methyl, ethyl, n-propyl, or 1-methylethyl-. The term “Cn+” compound or group, wherein n is a positive integer, means a compound or a group comprising carbon atoms therein at the number of equal to or greater than n. The term “Cn-” compound or group, wherein n is a positive integer, means a compound or a group comprising carbon atoms therein at the number of equal to or lower than n.

In the description herein, the catalyst may be described as a catalyst precursor, a pre-catalyst compound, or a catalyst compound, and these terms are used interchangeably.

The term “conversion” refers to the degree to which a given reactant in a particular reaction (e.g., methylation, isomerization, etc.) is converted to products. Thus 100% conversion of toluene to xylene in a methylation refers to complete consumption of the toluene, and 0% conversion of the toluene refers to no measurable reaction of the toluene.

The term “selectivity” refers to the degree to which a particular reaction forms a specific product, rather than another product. For example, for the methylation of toluene, 50% selectivity for p-xylene means that 50% of the products formed are p-xylene, and 100% selectivity for p-xylene means that 100% of the product formed is p-xylene. The selectivity is based on the product formed, regardless of the conversion of the particular reaction. The selectivity for a given product produced from a given reactant can be defined as weight percent (wt %) of that product relative to the total weight of the products formed from the given reactant in the reaction.

“Alkylation” means a chemical reaction in which an alkyl group is transferred to an aromatic ring as a substitute group thereon from an alkyl group source compound. “Methylation” means alkylation in which the transferred alkyl group is a methyl. Thus, methylation of benzene can produce toluene, xylenes, trimethylbenzenes, and the like; and methylation of toluene can produce xylenes, trimethylbenzenes, and the like. Toluene methylation with methanol in the presence of a zeolite catalyst can be schematically illustrated as follows

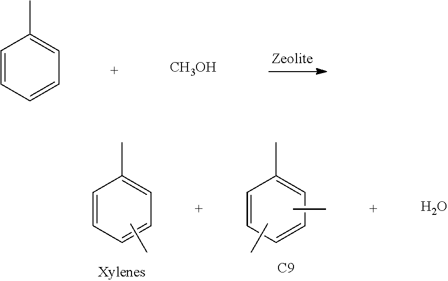

The xylenes include 1,2-dimethylbenzene (ortho-xylene, or o-xylene), 1,3-dimethylbenzene (meta-xylene, or m-xylene), and 1,4-dimethylbenzene (para-xylene, or p-xylene). One or more of these xylene isomers, particularly p-xylene and/or o-xylene, are high-value industrial chemicals. They can be separated to make corresponding products. The C9 hydrocarbons, though, are generally undesirable byproducts. The methylation reaction above can be performed in the presence of a zeolite catalyst.

As used herein, the term “molecular sieve” means a crystalline or semi-crystalline substance, e.g. a zeolite, with pores of molecular dimensions that permit the passage of molecules below a certain size.

In this disclosure, unless specified otherwise or the context clearly indicated otherwise. “space hourly weight velocity” is based on the combined flow rate of the aromatic hydrocarbon feed and the methylating agent feed.

The present disclosure describes staged systems and methods that use two or more catalyst fixed beds in series. The methylation reaction is an exothermic reaction where the increasing temperatures can cause catalyst degradation and the production of unwanted byproducts. By separating the catalyst into multiple beds and controlling the amount of methylating agent feed each catalyst bed is exposed to, the temperature in each bed may be more effectively controlled. Limiting the methylating agent feed may mitigate hot spots and runaway temperatures in catalyst beds because once the methylating agent feed is totally consumed, no further reaction occurs in a reactor. Then, the product stream from one catalyst bed can be then supplemented with an additional, controlled amount of methylating agent feed to provide similar temperature control advantages in the next catalyst bed. Further, the product stream from one catalyst bed can be cooled before introduction into the next catalyst bed, which can provides additional temperature control and may increase catalyst life and decrease production of unwanted byproducts. Accordingly, for the same amount of catalyst and reactants, performing methylation reactions using the staged systems and methods may produce a higher yield of xylenes, especially, p-xylenes while increasing catalyst lifetime.

### Methylation Systems and Processes

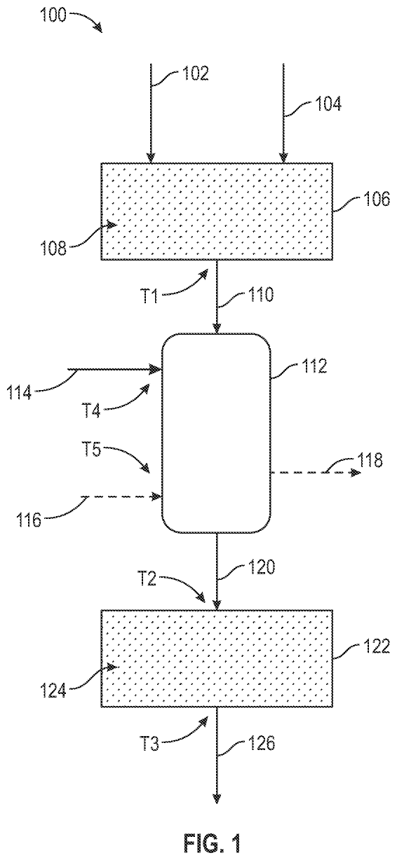

FIG. 1 illustrates a flow diagram of anon-limiting example method 100 of the present disclosure. The method 100 includes contacting a first aromatic hydrocarbon feed 102 (e.g., comprising benzene and/or toluene) and a first methylating agent feed 104 (e.g., comprising methanol and/or dimethyl ether) in the presence of a first methylation catalyst 108 that is in a first fixed bed 106. The first aromatic hydrocarbon feed 102 and the first methylating agent feed 104 may be introduced to the first fixed bed 106 independently or premixed and via one or more inlets.

The first fixed bed 106 may be under methylation conditions (e.g., temperatures of 200° C. to 500° C. and pressures of 100 kPa to 8500 kPa, additional conditions described further herein). In the first fixed bed 106, components of the aromatic hydrocarbon feed and the methylating agent feed may react to produce a first methylation product mixture 110 that comprises xylenes (e.g., p-xylene, o-xylene, and/or m-xylene). The temperature of the first methylation product mixture 110 exiting the first fixed bed may be temperature T1.

The first methylation product mixture 110 may then be treated 112 to produce a mixture feed 120. Treating 112 includes reducing a temperature of the first methylation product mixture 110 and introducing a second methylating agent feed 114 having a temperature T4 into the first methylation product mixture 110. Treating 112 may optionally further include introducing a second aromatic hydrocarbon feed 116 having a temperature T5 into the first methylation product mixture 110 and/or removing water from the first methylation product mixture 110. That is, treating 112 the first methylation product mixture 110 to produce the mixture feed 120 includes.
(a) reducing a temperature of the first methylation product mixture 110;
(b) injecting a second methylating agent feed 114 having a temperature T4 into the first methylation product mixture 110:
optionally (c) injecting a second aromatic hydrocarbon feed 116 having a temperature T5 into the first methylation product mixture 110; and
optionally (d) removing at least a portion of water 118 from the first methylation product mixture 110.

The foregoing steps may be performed in any order where one or more of the steps may be further performed simultaneously. Further, steps (b), (c), and/or (d) may cause, or otherwise facilitate, step (a) to be performed where, for example, (i) the second methylating agent feed 114 and/or the second aromatic hydrocarbon feed 116 are at a lower temperature than the first methylation product mixture 110 and/or (ii) the method of removing water also removes heat from the first methylation product mixture 110.

Temperature T1 may range from 200° C. to 500° C., such as from 275° C. to 475° C., from 300° C. to 450° C., or from 250° C. to 400° C.

Temperature T2 may range from 100° C. to 450° C., such as from 175° C. to 375° C., from 200° C. to 350°, or from 250° C. to 400° C.

A temperature difference between T1 and T2 (i.e., T1 minus T2, T1−T2) may be 25° C.≤T1−T2≤200° C., such as 25° C.≤T1−T2≤75° C. or 50° C.≤T1−T2≤125° C., or 75° C.≤T1−T2≤150° C., or 125° C.≤T1−T2≤200° C.

Temperature T4 may range from 25° C. to 500° C., such as from 50° C. to 150° C., from 100° C. to 250° C. or from 200° C. to 400° C.

A temperature difference between T1 and T4 (i.e., T1 minus T4, T1−T4) may be 0° C.≤T1−T4≤300° C., such as 25° C.≤T1−T4≤75° C., or 50° C.≤T1−T4≤125° C., or 100° C.≤T1−T4≤200° C., or 150° C.≤T1−T4≤250° C., or 225° C.≤T1−T4≤300° C.

Temperature T5 may range from 25° C. to 500° C., such as from 50° C. to 150° C., from 100° C. to 250° C., or from 200° C. to 400° C.

A temperature difference between T1 and T5 (i.e., T1 minus T5, T1−T5) may be 0° C.≤T1−T5≤300° C., such as 25° C.≤T1−T5≤75° C., or 50° C.≤T1−T5≤100° C., or 75° C.≤T1−T≤200° C., or 150° C.≤T1−T5≤250° C., or 225° C.≤T1−T5≤300° C.

As indicated, steps (b), (c), and/or (d) may be used to effect step (a) in the process of treating 112 the first methylation product mixture 110. However, one or more of steps (b), (c), and (d) may not affect the temperature of the first methylation product mixture 110. Rather, other heat removal methods and/or apparatuses may be used. For example, a heat exchanger may be located downstream of the first fixed bed 106 so as to transfer (or otherwise remove) at least a portion of an amount of heat from the first methylation product mixture 110 by using a heat exchanger.

By way of non-limiting example, the step of treating 112 may comprise: injecting the second methylating agent feed 114 having a temperature T4 into the first methylation product mixture 110, where T4<T1 thereby simultaneously reducing temperature of the first methylation product mixture 110. The step of treating 112 may further comprise: removing at least a portion of water 118 from the first methylation product mixture 110 before or after injecting the second methylating agent feed 114, or after injecting the second methylating agent feed 114.

By way of another non-limiting example, the step of treating 112 may comprise: reducing temperature of the first methylation product mixture 110 by passing the first methylation product mixture 110 through a heat exchanger or similar apparatus; and then injecting the second methylating agent feed 114 into the first methylation product mixture 110, which may or may not reduce the temperature of the first methylation product mixture 110. The step of treating 112 may further comprise: removing at least a portion of water 118 from the first methylation product mixture 110 before passing the first methylation product mixture 110 through the heat exchanger, between passing the first methylation product mixture 110 through the heat exchanger and injecting the second methylating agent feed 114, or after injecting the second methylating agent feed 114.

By way of yet another non-limiting example, the step of treating 112 may comprise: injecting the second methylating agent feed 114 having a temperature T4 into the first methylation product mixture 110, where T4<T1 thereby simultaneously reducing temperature of the first methylation product mixture 110; and then further reducing temperature of the first methylation product mixture 110 by passing the first methylation product mixture 110 through a heat exchanger or similar apparatus. The step of treating 112 may further comprise: removing at least a portion of water 118 from the first methylation product mixture 110 before injecting the second methylating agent feed 114, between injecting the second methylating agent feed 114 and passing the first methylation product mixture 110 through the heat exchanger, or after passing the first methylation product mixture 110 through a heat exchanger.

By way of another non-limiting example, the step of treating 112 may comprise: injecting the second methylating agent feed 114 having a temperature T4 and the second aromatic hydrocarbon feed 116 having a temperature T5 into the first methylation product mixture 110, where T4<T1 and T5<T1 thereby simultaneously reducing temperature of the first methylation product mixture 110, and, optionally, then further reducing temperature of the first methylation product mixture 110 by passing the first methylation product mixture 110 through a heat exchanger or similar apparatus. The step of treating 112 may further comprise: removing at least a portion of water 118 from the first methylation product mixture 110 before injecting the second methylating agent feed 114 and the second aromatic hydrocarbon feed 116, between injecting the second methylating agent feed 114 and the second aromatic hydrocarbon feed 116 and passing the first methylation product mixture 110 through the heat exchanger, or after passing the first methylation product mixture 110 through a heat exchanger.

By way of yet another non-limiting example, the step of treating 112 may comprise: injecting the second methylating agent feed 114 having a temperature T4 into the first methylation product mixture 110, where T4 has a value to cause a simultaneous reduction in temperature of the first methylation product mixture 110; and injecting the second aromatic hydrocarbon feed 116 having a temperature T5 into the first methylation product mixture 110, where T5 has a value to cause a simultaneous reduction in temperature of the first methylation product mixture 110, wherein the two injecting steps may occur in either order. The step of treating 112 may further comprise: removing at least a portion of water 118 from the first methylation product mixture 110 before both injections, between the two injections, or after both injections.

By way of another non-limiting example, the step of treating 112 may comprise: removing at least a portion of water 118 from the first methylation product mixture 110; then reducing temperature of the first methylation product mixture 110 by passing the first methylation product mixture 110 through a heat exchanger or similar apparatus; and then injecting the second methylating agent feed 114 into the first methylation product mixture 110, which may or may not reduce the temperature of the first methylation product mixture 110.

Removing at least a portion of the water for a designated stream, in any embodiment where water removal is performed, may be achieved by (a) passing the stream over an absorbent, (b) removing the water from the stream using a separation tank, (c) distilling the stream, or (d) any combination of any of the foregoing.

After treating 112 the first methylation product mixture 110 to produce the mixture feed 120, the method 100 includes contacting a mixture feed 120 with a second methylation catalyst 124 that is in a second fixed bed 122. The mixture feed 120 may be introduced to the second fixed bed 122 via one or more inlets.

The second fixed bed 122 may be under methylation conditions (e.g., temperatures of 200° C. to 50) ° C. and pressures of 100 kPa to 8500 kPa, additional conditions described further herein). In the second fixed bed 122, components of the mixture feed 120 may react to produce a second methylation product mixture 126 that comprises xylenes (e.g., p-xylene, o-xylene, and/or m-xylene). The temperature of the second methylation product mixture 126 exiting the second fixed bed 122 may be temperature T3.

Temperature T3 may range from 200° C. to 500° C., such as from 275° C. to 475° C., from 300° C. to 450° C., or from 250° C. to 400° C.

The second methylation product mixture 126 may be the final product or may be further treated (e.g., as treating 112) to produce at second mixed feed that may be reacted under methylation reaction conditions in a third fixed bed. That is, while FIG. 1 illustrates only two fixed beds and one treating between the fixed beds, methods and systems of the present disclosure may include more than two fixed beds (e.g., 3 or more, or 3 to 10 or more) with treating steps between adjacent fixed beds in series.

The composition of the methylation catalysts, the methylation reaction conditions in each fixed bed, the volume and/or dimensions of the fixed beds, the treating between fixed beds, the composition of gases going into (feeds) each fixed bed, and the composition of the gases coming out of (product mixtures) each fixed bed are independent and do not necessarily need to be the same. For example, the first methylation catalyst 108 and the second methylation catalyst 124 may be the same or different. In another example, treating 112 of the first methylation product mixture 110 and treating of the second methylation product mixture 126 may be the same or different.

Further, the fixed beds in the methods and systems described herein may be in the one vessel or in multiple vessels where each vessel contains one or more fixed beds. For example, in a two fixed bed situation, the first fixed bed may be contained in a first vessel, and the second fixed bed may be contained in a second vessel separate from the first vessel in fluid communication with the first vessel. In another example, in a three fixed bed situation, the first fixed bed may be contained in a first vessel, and the second and third fixed beds may be contained in a second vessel separate from the first vessel in fluid communication with the first vessel. Alternatively, the first and second fixed beds may be contained in a first vessel, and the third fixed bed may be contained in a second vessel separate from the first vessel in fluid communication with the first vessel.

Any suitable refinery aromatic feed can be used as the source of the benzene and/or toluene. The aromatic hydrocarbon feed (e.g., the first aromatic hydrocarbon feed 102 and the second aromatic hydrocarbon feed 116, independently) may comprise benzene and/or toluene at a concentration ≥90 wt % (e.g., ≥92 wt %, ≥94 wt %, ≥95 wt %, ≥96 wt %, ≥98 wt %, or even ≥99 wt %) based on the total weight of the aromatic hydrocarbon feed. In some embodiments, the aromatic hydrocarbon feed may be pre-treated to remove catalyst poisons, such as nitrogen and sulfur-compounds.

The ratio of aromatic hydrocarbon feed to methylating agent feed is R(a/m) which is determined by the following equation:

$$R ( a / m ) = \frac{M ( tol ) + 2 \cdot M ( bz )}{M ( methanol ) + 2 \cdot M ( DME )}$$

where M(tol) is the moles of toluene in the aromatic hydrocarbon feed, M(bz) is the moles of benzene in the aromatic hydrocarbon feed, M(methanol) is the moles of methanol in the methylating agent feed, and M(DME) is the moles of dimethyl ether in the methylating agent feed. At introduction to a fixed bed, R(a/m) may be ≥2, ≥3, ≥4, ≥5, or ≥6, and ≤10, ≤9, ≤8, ≤7, ≤6, ≤5, or ≤4, or, e.g., in a range from 2 to 10 or from 6 to 10. For the purpose of producing xylenes, each benzene molecule needs to be methylated by two methanol molecules or one DME molecule, and each toluene by one methanol molecule or half a DME molecule. Over-methylation of benzene and/or toluene can result in the production undesirable C9+ aromatic hydrocarbons as byproducts. To prevent over-methylation, it is highly desirable that R(a/m) ≥1.5. Preferably 2≤R(a/m)≤5. More preferably 2≤R(a/m)≤4. The efficiency of the methylation process can be reduced at higher R(a/m), e.g., R(a/m)>5, due to large quantity of toluene/benzene present in the methylation reaction product mixture, which needs to be separated and recycled to the methylation unit.

A methylation process of this disclosure (or under methylation reaction conditions) can be advantageously conducted at relatively low temperatures, for example ≤500° C., such as ≤475° C., ≤450° C., ≤425° C., or ≤400° C. A process may be conducted at temperatures of ≥200° C., such as ≥250° C., or ≥300° C. in the a fixed bed which has been found to provide commercially viable methylation reaction rates. In terms of ranges, the process may be conducted at temperatures ranging from 200° C. to 500° C., such as from 275° C. to 475° C., from 300° C. to 450° C., or from 250° C. to 400° C. Such low-temperature reaction can be particularly utilized when a MWW framework type zeolite is present in the methylation catalyst. Such low-temperature reaction can be particularly advantageous in fixed beds of the methylation catalyst. The ability of the processes of this disclosure to be operated at low temperature carries many advantages, to name a few: higher energy efficiency, longer catalyst life, fewer species of byproducts, and small quantities of byproducts that otherwise would be produced at higher temperatures, compared to conventional benzene/toluene methylation processes at temperatures higher than 500° C.

Operating pressures in the fixed bed (or under methylation reaction conditions) can vary in a broad range, e.g., from ≥100 kPa, such as ≥1000 kPa, ≥1500 kPa, ≥2000 kPa, ≥3000 kPa, or ≥3500 kPa, to ≤8500 kPa, such as ≤7000 kPa, or ≤6000 kPa. For example, operating pressures may range from 700 kPa to 7000 kPa, e.g., from 1000 kPa to 6000 kPa, or from 2000 kPa to 5000 kPa. In at least one embodiment, the combination of a high pressure (e.g., a pressure from 1500 kPa to 4500 kPa or even closer to 8500 kPa) and a low temperature (e.g., a temperature from 250-500° C.), decreases the amount of light gases produced in the methylation reaction, and may also decrease the catalyst aging rate.

WHSV values based on total aromatic hydrocarbon feed and methylating agent feed under methylation reaction conditions can be in the range from, e.g., 0.5 hour⁻¹ to 50 hour⁻¹, such as from 5 hour⁻¹ to 15 hour⁻¹, from 1 hour⁻¹ to 10 hour⁻¹, or from 5 hour⁻¹ to 10 hour⁻¹, or from 6.7 hour⁻¹ to 10 hour⁻¹. In some embodiments, at least part of the aromatic hydrocarbon feed, the methylating agent feed and/or the methylation product mixture may be present in the fixed bed or effluent thereof in the liquid phase. As is described in more detail below, alteration of the WHSV may be desired in concert with changes in temperature in order to maintain desired conversion of benzene, toluene, methanol, and/or dimethyl ether.

The efficiency of a methylation reaction in each fixed bed of methylation catalyst may be affected by the pressure drop across the fixed bed. The pressure drop depends on various factors such as the path length, the methylation catalyst particle size, and pore size. A pressure drop that is too large may cause channeling through the catalyst bed, and poor efficiency. In some embodiments, the fixed bed has a cylindrical geometry with axial flows through the catalyst bed.

The various designs of the fixed bed may accommodate control of specific process conditions, e.g., pressure, temperature, and WHSV. The WHSV determines volume and residence time that may provide the desired conversion.

The product of the methylation reaction, the methylation product mixture from each fixed bed, can comprise xylenes, benzene and/or toluene (both residual and coproduced in the process). C9+ aromatic hydrocarbons, co-produced water, and unreacted methanol and DME. In some embodiments, p-xylene is present in the methylation product mixture at ≥30 wt %, e.g., ≥40 wt %, ≥50 wt %, or ≥60 wt %, based on the total weight of the methylation product mixture. m-Xylene may be present in the methylation product mixture at ≤30 wt %. e.g., ≤20 wt %, ≤15 wt %, or ≤10 wt %, based on the total weight of the methylation product mixture. In some embodiments, the ratio of a p-xylene to m-xylene (pX:mX) in the methylation product mixture can be greater than 2:1, such as greater than 3:1, greater than 5:1, or greater than 7:1. In some embodiments, the ratio of o-xylene to m-xylene (oX:mX) in the methylation product mixture can be greater than 1:1, such as greater than 2:1, greater than 3:1, or greater than 4:1. In some embodiments, the ratio of a combination of p-xylene and o-xylene to m-xylene (pX+oX:mX) in the methylation product mixture is greater than 2:1, such as greater than 3:1, greater than 5:1, greater than 8:1, or greater than 10:1. In some embodiments, the process is operated at sufficient WHSV so that only a portion of the methanol is reacted with the aromatic hydrocarbon feed and the methylation product mixture contains residual methanol and/or DME.

The temperature in the methylation reaction will affect by-product formation and a temperature lower than 50) ° C. may decrease light gas formation. In some embodiments, the methylation product mixture from each fixed bed, independently, may contain ≤10 wt %, such as ≤wt %, ≤2 wt %, ≤1 wt %, or is substantially free of light gases generated by methanol decomposition to ethylene or other olefins.

DME, methanol, and/or toluene can be recovered through a separation subsystem. The separation subsystem may include one or more separation units. The separation subsystem may include any suitable method for recovery of a DME-rich stream, a methanol-rich stream, and/or a toluene-rich stream from the final methylation product mixture after the last fixed bed. In some embodiments, the separation subsystem includes a first recycle channel. In some embodiments, the first recycle channel is in fluid communication with the methylation agent feed or a methylation unit inlet. In some embodiments, the separation subsystem includes a first separation unit, the first separation unit may separate an aqueous phase and an oil phase. In some embodiments, the separation subsystem includes a second separation unit, the second separation unit may separate a DME-rich stream from the oil phase. In some embodiments, the DME-rich stream flows through the first recycle channel which may be in fluid communication with the second separation unit and the methylation agent feed or a methylation unit inlet. In another embodiment the second separation unit separates an aromatics rich stream from the oil phase. In some embodiments, the separation subsystem includes a third separation unit. The third separation unit may separate the aromatics-rich stream into a toluene-rich stream and a xylenes-rich stream. In some embodiments, the toluene-rich stream flows through a second recycle channel to the methylation agent feed or a methylation unit inlet. In some embodiments, the separation subsystem includes a fourth separation unit, the fourth separation unit may separate the aqueous phase into a water-rich stream and a methanol-rich stream. In at least one embodiment, the methanol-rich stream flows through the third recycle channel which may be in fluid communication with the fourth separation unit and the methylation agent feed or a methylation unit inlet.

In some embodiments, the final methylation product mixture from the last fixed bed is separated into an aqueous phase and an oil phase in a first separation unit. The method of separating the aqueous phase from the oil phase can be accomplished by a coalescing plate separator, e.g., described in U.S. Pat. Nos. 4,722,800 and 5,068,035; a centrifugal separator, e.g., described in U.S. Pat. Nos. 4,175,040; 4,959,158; and 5,591,340; a hydrocyclone separator, e.g., described in U.S. Pat. Nos. 4,428,839; 4,927,536; and 5,667,686; or other suitable methods. In some embodiments, the oil phase of the methylation product mixture may contain at least 80 wt % xylenes. In some embodiments, the methylation product mixture comprising an aqueous phase and an oil phase enters a first separation unit; the aqueous phase, which is denser, settles to the bottom of an upstream chamber and can be drawn from the water drain tube down below. The oil phase, which is lighter, is located on top of the aqueous phase and can spill over a dividing wall to the downstream chamber where it can then be drawn from the bottom of the downstream chamber.

After separation of the aqueous phase, the oil phase may be fed to a second separation unit to separate a DME-rich stream, an aromatics-rich stream, and methane or other by-products. In some embodiments, the DME-rich stream may be fully or partially separated from other products and by-products to be recycled through the first recycling channel. In some embodiments, the DME-rich stream contains DME in ≥50 wt %, ≥60 wt %, ≥70 wt %, ≥80 wt %, ≥90 wt %, ≥95 wt %, ≥98 wt %, or ≥99 wt %, based on the total weight of the DME-rich stream. In some embodiments, the methylating agent feed contains DME from the DME-rich stream in ≥20 wt %, ≥40 wt %, ≥60 wt %, ≥80 wt %, ≥90 wt %, ≥95 wt %, ≥98 wt %, or ≥99 wt %, based on the total weight of the DME in the methylating agent stream. In at least one embodiment all of the DME in the methylation agent feed is obtained from the DME-rich stream.

In some embodiments, the second separation unit partially or fully separates methane from other products, and by-products. In at least one embodiment, the methane is used as fuel gas.

In some embodiments, the second separation unit produces an aromatics-rich stream comprising C6 to C9+ aromatic hydrocarbon products and by-products. In another embodiment, the second separation unit produces a stream of C9+ aromatics. In at least one embodiment, the stream of C9+ aromatics can be recovered for blending into the gasoline pool or transalkylated with benzene and/or toluene to make additional xylenes. In some embodiments, the second separation unit produces a aromatics-rich stream comprising xylenes in ≥50 wt %, ≥60 wt %, ≥70 wt %, ≥80 wt %, ≥90 wt %, ≥95 wt %, ≥98 wt %, or ≥99 wt %, based on the total weight of the aromatics-rich stream. In some embodiments, the aromatics-rich stream comprises p-xylene. In some embodiments, the aromatics-rich stream contains p-xylene in ≥50 wt %, ≥60 wt %, ≥70 wt %, ≥80 wt %, ≥90 wt %, ≥95 wt %, ≥98 wt %, or ≥99 wt %, based on the total weight of the aromatics-rich stream.

In some embodiments the second separation unit is a distillation system comprising one or more distillation columns. The distillation system may be operated at increased pressure, such as ≥400 kPag, ≥500 kPag, ≥600 kPag, ≥700 kPag, ≥800 kPag, ≥900 kPag, such as from 400 kPag to 1400 kPag, from 600 kPag to 1300 kPag, from 700 kPag to 1200 kPag, from 800 kPag to 1100 kPag, or from 900 kPag to 1000 kPag.

In some embodiments, the aromatics-rich stream is processed in a third separation unit and further separated into a xylenes-rich stream and a toluene-rich stream, which may comprise benzene. The toluene-rich stream comprising benzene and/or toluene (to be recycled through a second recycling channel) may contain toluene in ≥50 wt %, ≥60 wt %, ≥70 wt %, ≥80 wt %, ≥90 wt %, ≥95 wt %, ≥98 wt %, or ≥99 wt %, based on the total weight of the toluene-rich stream. In another embodiment, the toluene-rich stream comprises benzene and toluene in a combined wt % of ≥50 wt %, ≥60 wt %, ≥70 wt %, ≥80 wt %, ≥90 wt %, ≥95 wt %, ≥98 wt %, or ≥99 wt %, based on the total weight of the toluene-rich stream. In some embodiments, the xylenes-rich stream contains an equilibrium mixture of ortho-, meta-, para-xylenes comprising about 24 wt % of p-xylene, about 50 wt % of meta-xylene, and about 26 wt % of ortho-xylene. The xylenes-rich stream may contain p-xylene in ≥10 wt %, ≥20 wt %, ≥30 wt %, ≥40 wt %, ≥50 wt %, ≥60 wt %, ≥70 wt %, or ≥80 wt %, based on the total weight of the xylenes-rich stream.

The xylenes-rich stream and one or more downstream C9+ transalkylation process streams may be sent to xylenes-loop to recover a p-xylene product and an optional o-xylene product. A xylenes loop can comprise a p-xylene recovery unit, such as a crystallization separation unit and/or an adsorptive chromatography separation unit known in the prior art. The p-xylene recovery unit can produce a high-purity p-xylene product and a p-xylene-deleted stream rich in o-xylene and m-xylene. The xylenes-loop can further comprise an isomerization unit such as a vapor-phase isomerization unit and/or a liquid phase isomerization unit known in the prior art to further convert a portion of the o-xylene and m-xylene in the p-xylene-depleted stream to p-xylene. The isomerized stream can be recycled to the p-xylene recovery unit in the xylenes loop to recover additional quantity of p-xylene.

In certain embodiments, the aqueous phase is transferred to a fourth separation unit to separate a methanol-rich stream from a water-rich stream. In some embodiments, the methanol-rich stream to be recycled through the third recycling channel contains methanol at ≥50 wt %, ≥60 wt %, ≥70 wt %, ≥80 wt %, ≥90 wt %, ≥95 wt %, ≥98 wt %, or ≥99 wt %, based the overall weight of the methanol-rich stream. In some embodiments the fourth separation unit is a distillation system, an example system is described in U.S. Pat. Nos. 3,293,154 and 4,210,495. In other embodiments the separation system employed is a membrane separation system or pervaporation separation system.

In another embodiment, the DME-rich stream is combined with the methanol-rich stream to form a single recycle stream. In another embodiment, the toluene-rich stream, the DME-rich stream, and the methanol-rich stream are combined to form a single recycle stream.

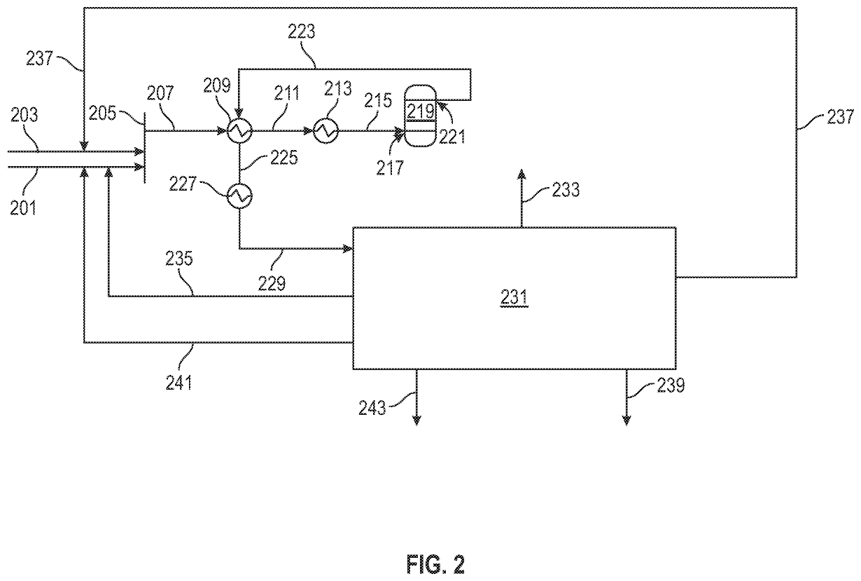

FIG. 2 schematically illustrates a process for converting benzene and/or toluene via methylation with methanol and/or DME to produce p-xylene according to one embodiment of this disclosure. Methylating agent feed 201, comprising methanol and/or DME is combined with aromatic hydrocarbon feed 203 comprising toluene and/or benzene in fluid transfer line 205. Fluid transfer line 205 may contain an agitator or other mixing device (not shown) in order to combine methylating agent feed 201 and aromatic hydrocarbon feed 203 to form a combined feed. The combined feed is fed by line 207 to heat exchanger 209 to pre-heat the combined feed. The heated combined feed comprising a mixture of feed 201 and feed 203 is fed through line 211 to heat exchanger 213. Heat exchanger 213 may be used to heat or cool the combined feed as necessary. The combined feed is then passed through line 215, through inlet 217 to methylation unit 219. Line 215 may also include a pump or series of pumps (not shown) in order to maintain sufficient pressure and WHSV in methylation unit 219. Inlet 217 may accept one or more feeds or streams comprising one or more recycle streams. Methylation unit 219 includes staged fixed beds described herein (e.g., comprising two or more fixed beds in series, comprising additional gas inlets (not shown), lines (not shown), pumps (not shown), and optionally heat exchangers (not shown) for executing the staged methods described herein), each fixed bed independently containing the methylation catalyst (not shown) and operated at methylation reaction conditions, which may include a temperature lower than 500° C. and an absolute pressure ≥100 kPa. The product of the staged fixed beds in the methylation unit (the final methylation product mixture effluent) can be a mixture of xylenes, water, methanol, dimethyl ether, and by-products and is fed from methylation unit 219 through outlet 221 to line 223 and ultimately to heat exchanger 209 to be cooled. The cooled methylation product mixture effluent is passed through line 225 to heat exchanger 227 to be either heated or cooled as necessary to arrive at the desired temperature for separation, then through line 229 to separation subsystem 231. Separation subsystem 231 may contain one or more separation units (not shown). Separation subsystem 231 may separate methane or other light gases which can be removed via line 233 and may be used as fuel gas (not shown).

Separation subsystem 231 may further separate a dimethyl ether-rich stream which is then provided to line 235, which can be recycled into methylating agent feed 201 or methylation unit inlet 217. Line 235 may include pumps or compressors so that the DME-rich stream may enter the methylation agent feed or methylation unit at a desired pressure, the combination of lines and pumps or compressors is a first recycling channel. The first recycling channel may contain other combinations of lines and pumps or compressors (not shown) suitable to recycle DME to methylation unit 219.

Separation subsystem 231 may further separate toluene-rich stream 237, which may contain benzene and can be recycled into aromatic hydrocarbon feed 203 or methylation unit inlet 217. Line 237 may include pumps or compressors so that the toluene-rich stream may enter the aromatic hydrocarbon feed or methylation unit or fixed bed at a desired pressure; the combination of lines and pumps or compressors is a second recycling channel. Furthermore, the separation may yield a xylenes-rich stream which is sent out of line 239, and line 239 may be connected to other systems for further processing (not shown). The xylenes-rich stream can be fed to a separation system such as a crystallizer or a simulated moving bed adsorption chromatography to recover a high-purity p-xylene product and produce a p-xylene-depleted stream. The p-xylene-depleted stream can be isomerized in an isomerization reactor in the presence of an isomerization catalyst to produce additional p-xylene.

Separation subsystem 231 may further separate a methanol-rich stream which is then provided to line 241, which can be recycled into methylating agent feed 201 or methylation unit inlet 217. Line 241 may include pumps or compressors so that the methanol-rich stream may enter the methylation agent feed or methylation unit or fixed bed at a desired pressure; the combination of lines and pumps or compressors is a third recycling channel. The third recycling channel may contain other combinations of lines and pumps or compressors (not shown) suitable to recycle methanol to methylation unit 219. Furthermore, the separation may yield a water-rich stream which is sent out of line 243, and line 243 may be connected to other systems for further processing (not shown), comprising wastewater purification systems (not shown).

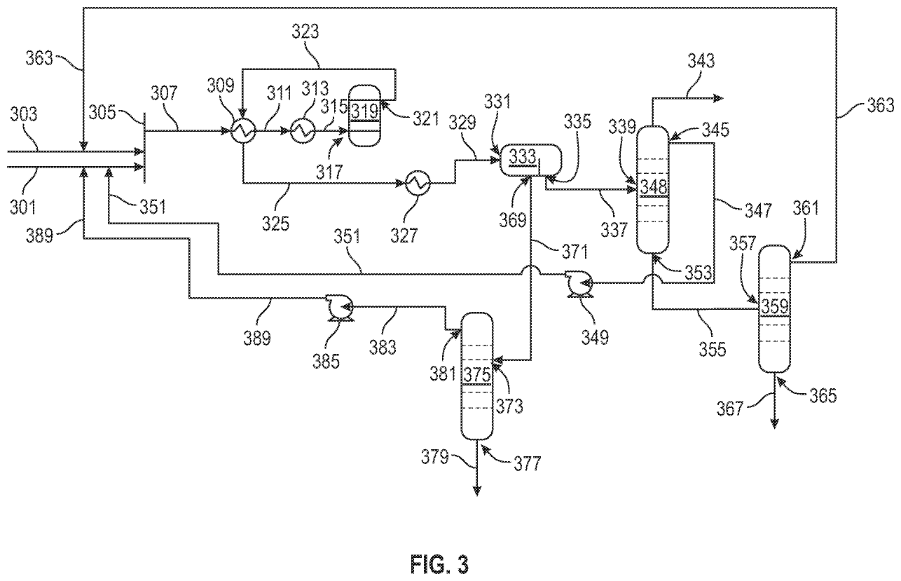

FIG. 3 schematically illustrates a process for converting benzene/toluene via methylation with methanol/dimethyl ether to produce p-xylene according to an embodiment of this disclosure. Methylating agent feed 301, comprising methanol and/or DME, is combined with aromatic hydrocarbon feed 303 comprising toluene and/or benzene in fluid transfer line 305. Fluid transfer line 305 may contain an agitator or other mixing device (not shown) in order to fully combine methylating agent feed 301 and aromatic hydrocarbon feed 303. The combined feed is transferred by line 307 to heat exchanger 309 to pre-heat the combined feed. The heated combined feed comprising a mixture of feed 301 and feed 303 is fed through line 311 to heat exchanger 313. Heat exchanger 313 may be used to heat or cool the combined feed as necessary. The combined feed is then passed through line 315, through inlet 317, to methylation unit 319. Line 315 may also include a pump or series of pumps (not shown) in order to maintain sufficient pressure and WHSV in methylation unit 319. Inlet 317 may accept one or more feeds or streams comprising one or more recycle streams. Methylation unit 319 includes staged fixed beds described herein (e.g., comprising two or more fixed beds in series, comprising additional gas inlets (not shown), lines (not shown), pumps (not shown), and optionally heat exchangers (not shown) for executing the staged methods described herein), each fixed bed independently containing the methylation catalyst (not shown) and operated at methylation reaction conditions, which may include a temperature lower than 500° C. and an absolute pressure ≥100 kPa. Methylation unit 319 contains two or more fixed beds (not shown) where methylation catalyst is present. The final product of the methylation conditions in the methylation unit (the final methylation product mixture effluent) can be a mixture of xylenes, water, methanol, dimethyl ether, and by-products. The methylation product mixture effluent is transferred from methylation unit 319 through outlet 321 to line 323 leading to heat exchanger 309 to be cooled, the cooled methylation product mixture effluent is passed through line 325 to heat exchanger 327 to be either heated or cooled as necessary to arrive at the desired temperature for separation, then through line 329 to inlet 331 of first separation unit 333.

First separation unit 333 separates the aqueous phase (a water/methanol mixture) of the methylation product mixture effluent from the oil phase (a hydrocarbon portion of the methylation product mixture effluent) of the methylation product mixture effluent. First separation unit 333 may function through any suitable method of separating aqueous and oil phases, including simple phase separation, hydrocyclonic separation, or other suitable methods. The oil phase of the methylation product mixture effluent may contain xylenes, methane, dimethyl ether, unreacted benzene or toluene, and other by-products. The hydrocarbon portion of the methylation product mixture effluent is passed through outlet 335 through line 337 to inlet 339 of second separation unit 348. The aqueous phase is passed through outlet 369 to line 371.

Second separation unit 348 separates the oil phase into (i) a light gas portion comprising methane, which can be vented to fuel gas through line 343; (ii) a dimethyl ether-rich stream, which is passed through outlet 345 through line 347 to pump 349, through line 351 and is recycled into methylating agent feed 301 or methylation unit inlet 317; the combination of lines and pumps or compressors is a first recycling channel; and (iii) a aromatics-rich stream comprising p-xylene, which can be removed for further processing through outlet 353 and line 355. Second separation unit 348 can be a distillation column run at sufficient pressure to allow dimethyl ether to be separated as a liquid while not requiring a bottoms temperature sufficiently high so as to cause decomposition of portions of the methylation product mixture effluent.

The aromatics-rich stream passed though line 355 may be introduced to inlet 357 and into third separation unit 359 where it can be separated. The separation may yield a toluene-rich stream which is sent out of outlet 361 through line 363 and may be recycled to aromatic hydrocarbon feed 303 or methylation unit inlet 317. Line 363 may include pumps or compressors so that the toluene-rich stream may enter the aromatic hydrocarbon feed or methylation unit or fixed bed at a desired pressure, the combination of lines and pumps or compressors is a second recycling channel. Furthermore, the separation may yield a xylenes-rich stream which is sent out of outlet 365 through line 367, and line 367 may be connected to other systems for further processing (not shown).

The aqueous phase from first separation unit 333 can be passed through outlet 369 and through line 371 to inlet 373 of fourth separation unit 375. Fourth separation unit 375 separates a water-rich stream and a methanol-rich stream. Fourth separation unit 375 can function through any suitable method of separating methanol and water, including distillation, pervaporation, membrane separation, or other suitable methods. The water rich stream may be passed through outlet 377 and line 379 for further processing or disposal. The methanol rich stream can be sent out of outlet 381 to line 383 and to pump 385. The methanol rich stream may then be passed through line 389 and introduced to methylating agent feed 301 or methylation unit inlet 317 (not shown). Lines 383 and 389 may contain additional pumps (additional to pump 385) or compressors to return the methanol-rich stream at a desired pressure to either methylation agent feed 301 or methylation unit inlet 317; the combination of lines and pumps are a third recycle channel.

### Methylation Catalyst

Any suitable catalyst capable of converting toluene (or benzene) to xylenes can be used for the methylation process of this disclosure. Examples of such catalysts are crystalline microporous materials including zeolite-based, as well as non-zeolite-based, molecular sieves and can be of the large, medium, or small pore type. Molecular sieves can have 3-dimensional, four-connected framework structure of corner-sharing [TO₄] tetrahedra, where T can be a tetrahedrally coordinated atom. These molecular sieves are often described in terms of the size of the ring that defines a pore, where the size is based on the number of T atoms in the ring. Other framework-type characteristics include the arrangement of rings that form a cage, and, when present, the dimension of channels, and the spaces between the cages. See van Bekkum, et al, Introduction to Zeolite Science and Practice, Second Completely Revised and Expanded Edition, Volume 137, pages 1-67, Elsevier Science, B.V., Amsterdam, Netherlands (2001). Another convenient measure of the extent to which a molecular sieve provides control of molecules of varying sizes to its internal structure is the Constraint Index. The method by which Constraint Index is determined is described fully in U.S. Pat. No. 4,016,218, which is incorporated herein by reference for details of the method.

Non-limiting examples of molecular sieves include small pore molecular sieves (e.g., AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, THO, and substituted forms thereof), medium pore molecular sieves (e.g., AFO, AEL, EUO, HEU, FER, MEL, MFI, MTW, MTT, TON, and substituted forms thereof), large pore molecular sieves (e.g., EMT, FAU, and substituted forms thereof), intergrowths thereof, and combinations thereof. Other molecular sieves include, but are not limited to, ANA, BEA, CFI, CLO, DON, GIS, LTL, MER, MOR, MWW, SOD, intergrowths thereof, and combinations thereof. In some embodiments, the molecular sieve has an MWW framework type (morphology).

The small, medium, and large pore molecular sieves have from a 4-ring to a 12-ring or greater framework-type. In some embodiments, the zeolitic molecular sieves have 6-, 8-, 10-, or 12-ring structures and an average pore size in the range from about 3 Å to 15 Å. In other embodiments, the molecular sieves are aluminosilicate molecular sieves and have a 6-ring or an 8-ring structure and an average pore size of about 5 Å or less, such as in the range from 3 Å to about 5 Å, for example from 3 Å to about 4.5 Å or from 3.5 Å to about 4.2 Å.

Other non-limiting examples of zeolitic and non-zeolitic molecular sieves include one or a combination of the following: Beta (U.S. Pat. No. 3,308,069 and Reissue No. 28,341), ZSM-3 (U.S. Pat. No. 3,415,736), ZSM-4 (U.S. Pat. No. 4,021,947), ZSM-5 (U.S. Pat. Nos. 3,702,886, 4,797,267 and 5,783,321), ZSM-11 (U.S. Pat. No. 3,709,979), ZSM-12 (U.S. Pat. No. 3,832,449), ZSM-12 and ZSM-38 (U.S. Pat. No. 3,948,758), ZSM-14 (U.S. Pat. No. 3,923,636), ZSM-18 (U.S. Pat. No. 3,950,496), ZSM-20 (U.S. Pat. No. 3,972,983), ZSM-22 (U.S. Pat. No. 5,336,478), ZSM-23 (U.S. Pat. No. 4,076,842), ZSM-34 (U.S. Pat. No. 4,086,186), ZSM-35 (U.S. Pat. No. 4,016,245), ZSM-38, ZSM-48 (U.S. Pat. No. 4,397,827), ZSM-50, ZSM-58 (U.S. Pat. No. 4,698,217), MCM-1 (U.S. Pat. No. 4,639,358). MCM-2 (U.S. Pat. No. 4,673,559). MCM-3 (U.S. Pat. No. 4,632,811), MCM-4 (U.S. Pat. No. 4,664,897), MCM-5 (U.S. Pat. No. 4,639,357), MCM-9 (U.S. Pat. No. 4,880,611), MCM-10 (U.S. Pat. No. 4,623,527). MCM-14 (U.S. Pat. No. 4,619,818), MCM-22 (U.S. Pat. No. 4,954,325). MCM-41 (U.S. Pat. No. 5,098,684), M-41S (U.S. Pat. No. 5,102,643), MCM-48 (U.S. Pat. No. 5,198,203), MCM-49 (U.S. Pat. No. 5,236,575), MCM-56 (U.S. Pat. No. 5,362,697), ALPO-11 (U.S. Pat. No. 4,310,440), ultrastable Y zeolite (USY) (U.S. Pat. Nos. 3,293,192 and 3,449,070), Dealuminized Y zeolite (Deal Y) (U.S. Pat. No. 3,442,795), mordenite (naturally occurring and synthetic) (for synthetic mordenite U.S. Pat. Nos. 3,766,093 and 3,894,104), SSZ-13, titanium aluminosilicates (TASOs) such as TASO-45 (European Patent No. EP-A-0 229 295), boron silicates (U.S. Pat. No. 4,254,297), titanium aluminophosphates (TAPOs) (U.S. Pat. No. 4,500,651), mixtures of ZSM-5 and ZSM-11 (U.S. Pat. No. 4,229,424), ECR-18 (U.S. Pat. No. 5,278,345), SAPO-34 bound ALPO-5 (U.S. Pat. No. 5,972,203), those disclosed in International Publication No. WO 98/57743 published Dec. 23, 1988 (molecular sieve and Fischer-Tropsch), those disclosed in U.S. Pat. No. 6,300,535 (MFI-bound zeolites), mesoporous molecular sieves (U.S. Pat. Nos. 6,284,696, 5,098,684, 5,102,643 and 5,108,725), and the like, and intergrowths and/or combinations thereof.

In an embodiment, the methylation catalyst comprises an aluminosilicate catalyst composition. Aluminosilicates, as used herein, can include those having a molar relationship of X₂O₃:(n)YO₂ (wherein X is a trivalent element, e.g., Al; and Y is a tetravalent element, e.g., Si), in which n≤500, such as ≤250, ≤100, such as from 30 to 100.

Non-limiting examples of trivalent X can include aluminum, boron, iron, indium, gallium, and combinations thereof, for example X can be aluminum. Non-limiting examples of tetravalent Y can include silicon, tin, titanium, germanium, and combinations thereof, for example Y can be silicon.

Other non-limiting examples of aluminosilicate catalysts and compositions can be found, for instance, in U.S. Patent Application Publication No. 2003/0176751 and U.S. patent application Ser. No. 11/017,286 (filed Dec. 20, 2004) and 60/731,846 (filed Oct. 31, 2005).

One class of molecular sieve suitable for use in a process of this disclosure has a Constraint Index ≤5, and is crystalline microporous material of the MWW framework type. In at least one embodiment, the crystalline microporous material is a zeolite. As used herein, the term “crystalline microporous material of the MWW framework type” includes one or more of:
(a) molecular sieves made from a common first degree crystalline building block unit cell, which unit cell has the MWW framework topology. (A unit cell is a spatial arrangement of atoms which if tiled in three-dimensional space describes the crystal structure. Such crystal structures are discussed in the “Atlas of Zeolite Framework Types”, Fifth edition, 2001, incorporated herein by reference);
(b) molecular sieves made from a second degree building block, being a 2-dimensional tiling of such MWW framework topology unit cells, forming a monolayer of one unit cell thickness, in an embodiment, one c-unit cell thickness;
(c) molecular sieves made from common second degree building blocks, being layers of one or more than one unit cell thickness, where the layer of more than one unit cell thickness is made from stacking, packing, or binding at least two monolayers of MWW framework topology unit cells. The stacking of such second degree building blocks can be in a regular fashion, an irregular fashion, a random fashion, or any combination thereof, and
(d) molecular sieves made by any regular or random 2-dimensional or 3-dimensional combination of unit cells having the MWW framework topology.

Crystalline microporous materials of the MWW framework type include those molecular sieves having an X-ray diffraction pattern comprising d-spacing maxima at 12.4±0.25, 6.9±0.15, 3.57±0.07 and 3.42±0.07 Angstrom. The X-ray diffraction data used to characterize the material are obtained by standard techniques using the K-alpha doublet of copper as incident radiation and a diffractometer equipped with a scintillation counter and associated computer as the collection system.

Examples of crystalline microporous materials of the MWW framework type include MCM-22 (U.S. Pat. No. 4,954,325), PSH-3 (U.S. Pat. No. 4,439,409). SSZ-25 (U.S. Pat. No. 4,826,667), ERB-1 (European Patent No. 0293032), ITQ-1 (U.S. Pat. No. 6,077,498), ITQ-2 (International Publication No. WO97/17290), MCM-36 (U.S. Pat. No. 5,250,277), MCM-49 (U.S. Pat. No. 5,236,575), MCM-56 (U.S. Pat. No. 5,362,697). UZM-8 (U.S. Pat. No. 6,756,030), UZM-8HS (U.S. Pat. No. 7,713,513), UZM-37 (U.S. Pat. No. 7,982,084), EMM-10 (U.S. Pat. No. 7,842,277). EMM-12 (U.S. Pat. No. 8,704,025), EMM-13 (U.S. Pat. No. 8,704,023), UCB-3 (U.S. Pat. No. 9,790,143B2) and mixtures thereof.

In some embodiments, the crystalline microporous material of the MWW framework type may be contaminated with other crystalline materials, such as ferrierite or quartz. These contaminants may be present in quantities of ≤10 wt %, such as ≤5 wt %.

In some embodiments, the molecular sieves are not subjected to pre-treatments, such as high temperature steaming, to modify their diffusion properties. In other embodiments, the molecular sieves may be selectivated, either before introduction into the aromatization reactor or in-situ in the reactor, by contacting the catalyst with a selectivating agent, such as silicon, steam, coke, or a combination thereof. In one embodiment, the catalyst is silica-selectivated by contacting the catalyst with at least one organosilicon in a liquid carrier and subsequently calcining the silicon-containing catalyst in an oxygen-containing atmosphere, e.g., air, at a temperature of 350° C. to 550° C. A suitable silica-selectivation procedure is described in U.S. Pat. No. 5,476,823. In another embodiment, the catalyst is selectivated by contacting the catalyst with steam. Steaming of the zeolite is effected at a temperature of ≥950° C., such as from 950° C. to 1075° C., or from 1000° C. to 1050° C., for 10 minutes to 10 hours, such as from 30 minutes to 5 hours. The selectivation procedure, which may be repeated multiple times, alters the diffusion characteristics of the molecular sieve and may increase the xylene yield.

In addition to, or in place of, silica or steam selectivation, the catalyst may be subjected to coke selectivation. This optional coke selectivation typically involves contacting the catalyst with a thermally decomposable organic compound at an elevated temperature in excess of the decomposition temperature of said compound but below the temperature at which the crystallinity of the molecular sieve is adversely affected. Further details regarding coke selectivation techniques are provided in the U.S. Pat. No. 4,117,026. In some embodiments, a combination of silica selectivation and coke selectivation may be employed.

It may be desirable to combine the molecular sieve, prior to selectivating, with at least one oxide modifier, such as at least one oxide selected from elements of Groups 2 to 4 and 13 to 16 of the Periodic Table. In some embodiments, the oxide modifier is selected from oxides of boron, magnesium, calcium, lanthanum, and phosphorus. In some cases, the molecular sieve may be combined with more than one oxide modifier, for example a combination of oxides of phosphorus with calcium and/or magnesium, since in this way it may be possible to reduce the steaming severity needed to achieve a target diffusivity value. In some embodiments, the total amount of oxide modifier present in the catalyst, as measured on an elemental basis, may be from 0.05 wt % and 20 wt %, such as from 0.1 wt % to 10 wt %, based on the weight of the final catalyst. Where the modifier comprises phosphorus, incorporation of modifier into the catalyst is conveniently achieved by the methods described in U.S. Pat. Nos. 4,356,338; 5,110,776; 5,231,064; and 5,348,643.

The molecular sieve may be formulated into the methylation catalyst without any binder, in a self-bound form. Alternatively, the molecular sieve may be formulated with a binder material to produce the methylation catalyst. A binder can be resistant to the temperatures and other conditions employed in the methylation reaction. That is, the methylation catalyst may comprise (a) zeolites having MWW framework type, and optionally (b) binder. Such binder materials may be active or inactive. Such binder materials may be synthetic and/or naturally occurring. Non-limiting examples of useful binders are clay (e.g., bentonite, kaolin), oxides such as alumina, silica, silica-alumina, zirconia, titania, magnesia, and mixtures, combinations, and composites thereof. The binders can impart desirable mechanical properties such as crush strength to and/or aid in forming (e.g., extrusion) the precursor catalyst. Inactive binder materials also serves the function of a diluent of the active zeolite. In certain embodiments, the methylation catalyst can comprise from 10 wt %, 20 wt %, 30 wt %, to 40 wt %, 50 wt %, 60 wt %, to 70 wt %, 80 wt %, or 90 wt %, of the MWW framework zeolite, based on the total weight of the precursor catalyst, and the remainder being a binder material. In addition to the MWW framework zeolite, the methylation catalyst may optionally comprise other zeolite as well.

The molecular sieves may be used as the methylation catalyst without any binder or matrix, in a self-bound form. Alternatively, the molecular sieves may be composited with another material which is resistant to the temperatures and other conditions employed in the methylation reaction. Such binder or matrix materials include active and inactive materials and synthetic or naturally occurring zeolites as well as inorganic materials such as clays and/or oxides such as alumina, silica, silica-alumina, zirconia, titania, magnesia or mixtures of these and other oxides. The latter may be either naturally occurring or in the form of gelatinous precipitates or gels comprising mixtures of silica and metal oxides. Clays may also be included with the oxide type binders to modify the mechanical properties of the catalyst or to assist in its manufacture. Use of a material in conjunction with the molecular sieve whether combined therewith or present during its synthesis, which itself is catalytically active may change the conversion and/or selectivity of the catalyst. Inactive materials suitably serve as diluents to control the amount of conversion so that products may be obtained economically and orderly without employing other means for controlling the rate of reaction. These materials may be incorporated into naturally occurring clays, for example, bentonite and kaolin, to improve the crush strength of the catalyst under commercial operating conditions and function as binders or matrices for the catalyst. The relative proportions of molecular sieve and inorganic oxide matrix vary widely, with the sieve content ranging from 1 wt % to 90 wt %, and in some embodiments the composite is prepared in the form of beads, in the range of 2 wt % to 80 wt % of the composite.

To facilitate a better understanding of the embodiments of the present disclosure, the following examples of preferred or representative embodiments are given. In no way should the following examples be read to limit, or to define, the scope of the invention.

### EXAMPLES

Using a kinetic model developed for the process via Athena Virtual Studio, a single fixed bed design was compared to that of a two fixed beds in series design. The inlet for all fixed beds was kept the same, either 260° C. or 280° C. Run 1 was a single fixed bed simulation with a 260° C. inlet, which can be compared to Run 3, a two fixed beds in series simulation also with 260° C. inlets for each fixed bed. Run 2 was a single fixed bed simulation with a 280° C. inlet, which can be compared to Run 4, a two fixed beds in series simulation also with 280° C. inlets for each fixed bed. For Runs 3 and 4, methanol was also before each of the two fixed beds. Each Run uses the same total amount of methanol. However, in Runs 3 and 4 half of the total methanol was sent into the first fixed bed and the remaining methanol into the second fixed bed. Table I is the results of the simulation.

TABLE 1

| | | | | |
|---|---|---|---|---|
| Conditions | Run 1 | Run 2 | Run 3 | Run 4 |

First Stage

| | | | | |
|---|---|---|---|---|
| Inlet Temp (° C.) | 260 | 280 | 260 | 280 |
| Outlet Temp (° C.) | 348 | 366 | 312 | 331 |
| WHSV (hr⁻¹) | 6 | 6 | 12 | 12 |
| MeOH:Toluene (mole ratio) | 0.33 | 0.33 | 0.167 | 0.167 |
| Toluene Conversion | 25.7% | 25.2 | 15.1% | 14.8% |
| MeOH Conversion | 100.0% | 100.0% | 100.0% | 100.0% |
| MeOH Utilization* | 100.0% | 100.0% | 99.9% | 99.9% |
| Xylene Selectivity | 84.8% | 80.2% | 62.2% | 62.2% |
| p-Xylene Selectivity | 57.6% | 55.0% | 62.2% | 62.2% |
| Benzene Selectivity | 0.1% | 0.1% | 0.1% | 0.1% |
| A9 Selectivity | 8.2% | 12.9% | 2.2% | 2.2% |
| A10+ Selectivity | 7.0% | 6.9% | 2.8% | 3.0% |

Second Stage

| | | | | |
|---|---|---|---|---|
| Inlet Temp (° C.) | n/a | n/a | 260 | 280 |
| Outlet Temp (° C.) | n/a | n/a | 303 | 321 |
| WHSV (hr⁻¹) | n/a | n/a | 12 | 12 |
| MeOH:Toluene | n/a | n/a | 0.167 | 0.167 |
| (mole ratio) | | | | |
| Toluene Conversion | n/a | n/a | 26.2% | 25.5% |
| MeOH Conversion | n/a | n/a | 100.0% | 100.0% |
| MeOH Utilization* | n/a | n/a | 99.9% | 99.9% |
| Xylene Selectivity | n/a | n/a | 91.7% | 88.5% |
| p-Xylene Selectivity | n/a | n/a | 60.9% | 59.4% |
| Benzene Selectivity | n/a | n/a | 0.1% | 0.1% |
| A9 Selectivity | n/a | n/a | 1.7% | 3.3% |
| A10+ Selectivity | n/a | n/a | 6.5% | 8.0% |

*MeOH Utilization is the % of methyl groups from methanol that are added to an aromatic ring.

Comparing Run 1 and Run 3, toluene conversion is increased and heavies (A9+'s) are reduced in the two fixed beds approach that includes introducing methanol and cooling between the two fixed beds. Additionally, both overall xylene selectivity and p-xylene selectivity over the other isomers, increased with the two fixed beds approach. Similar results are seen when comparing Run 2 to Run 4, which uses an inlet temperature of 280° C.

Comparing Runs 1 and 2 or Runs 3 and 4 shows the temperature effect on the process. At higher temperatures selectivity to xylenes and p-xylene selectivity is reduced, while selectivity to A9+'s is increased.

Based on the two foregoing comparisons, cooling between fixed beds and having a lower the overall temperature of the fixed bed, the reaction may favor the selectivity of p-xylene, the product of interest.

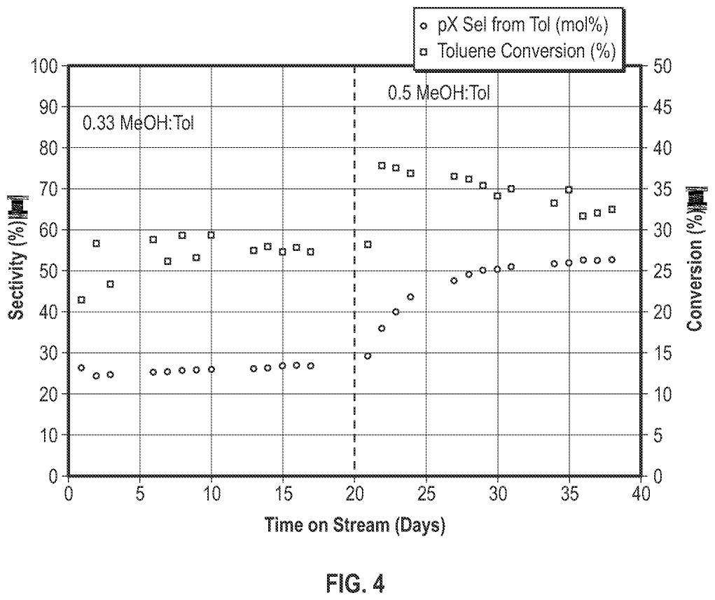

The kinetic model was also used to simulate Run 4 where after 20 days on stream the total MeOH:toluene mole ratio was increased to (0.25 MeOH:toluene mole ratio in for each fixed bed). FIG. 4 is a plot of the results. Higher methanol to toluene ratios going into each fixed bed increased selectivity to p-xylene.

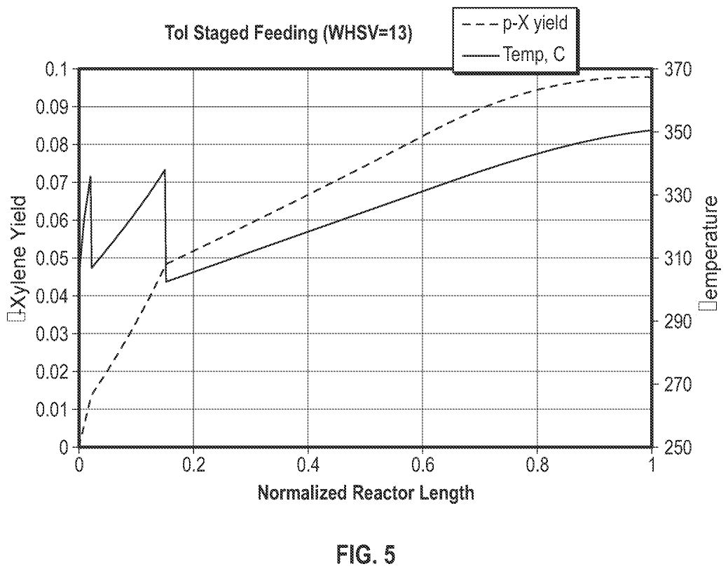

The kinetic model was also used to simulate another run where the toluene at a temperature lower than the reactor temperature is added to a single reactor at two different locations, which provides cooling to the system. The results are shown in FIG. 5. The inlet temperature of the reactor is 305° C. The first injection was with toluene at 270° C. (0.02 L) (located at first temperature drop in FIG. 5) and the second injection was at 220° C. (0.15 L) (located at second temperature drop in FIG. 5). Without the staged, injection of cooler toluene the temperature rise would be 90° C., but with the staged injections the temperature rise was only 40° C.

### NON-LIMITING EXAMPLE EMBODIMENTS

This disclosure can further include the following non-limiting embodiments.

A1: A process for producing p-xylene, the process comprising: (I) contacting an aromatic hydrocarbon feed with a first methylating agent feed in the presence of a first methylation catalyst in a first fixed bed under a first set of methylation reaction conditions to produce a first methylation product mixture comprising p-xylene exiting the first fixed bed, wherein the aromatic hydrocarbon feed comprises benzene and/or toluene, wherein the first methylating agent feed comprises methanol and/or dimethyl ether, and wherein the first methylation product mixture when exiting the first fixed bed has a first temperature of T1; (II) producing a mixture feed having a second temperature of T2 via steps comprising: (IIa) reducing a temperature of the first methylation product mixture and (IIb) injecting a second methylating agent feed having a fourth temperature T4 into the first methylation product mixture, wherein step (IIa) occurs before, after, and/or during step (IIb), and wherein the second methylating agent feed comprises methanol and/or dimethyl ether; and (III) contacting the mixture feed with a second methylation catalyst in a second fixed bed under a second set of methylation reaction conditions to produce a second methylation product mixture comprising p-xylene exiting the second fixed bed, wherein the second methylation product mixture when exiting the second fixed bed has a third temperature of T3.

A2: The process of A1, wherein 25° C.≤T1−T2≤200° C.

A3: The process of A1 or A2, wherein at least one of the following is met: 200° C.≤T1≤500° C.; and 200° C.≤T3≤500° C.

A4: The process of any of A1 to A3, wherein at least one of the following is met: 250° C.≤T1≤400° C. and 250° C.≤T3≤400° C.

A5: The process of any of A1 to A4, wherein 25° C.≤T1−T4≤300° C.

A6: The process of any of A1 to A5, wherein step (IIa) comprises: transferring at least a portion of an amount of heat from the first methylation product mixture by using a heat exchanger.

A7: The process of any of A1 to A6, wherein step (II) further comprises: (IIc) injecting a second aromatic hydrocarbon feed having a fifth temperature T5 into the first methylation product mixture, wherein the second aromatic hydrocarbon feed comprises benzene and/or toluene, and wherein step (IIa), step (IIb), and step (IIc) independently occur in any order and/or simultaneously.

A8: The process of A7, wherein 25° C.≤T1−T5≤300° C., and wherein the mixture feed comprises the first methylation product mixture, the second methylating agent feed, and the second aromatic hydrocarbon feed.

A9: The process of A7 or A8, wherein step (II) comprises: at least a portion of step (IIa) occurs simultaneous with at least a portion of step (lib).

A10: The process of A7 or A8, wherein step (II) comprises: step (IIa) before or after step (IIb).

A11: The process of A7 or A8, wherein step (II) comprises: at least a portion of step (IIc) occurs simultaneous with at least a portion of step (IIb).

A12: The process of A7 or A8, wherein step (II) comprises: step (IIc) before or after step (IIb).

A13: The process of A7 or A8, wherein step (II) comprises: at least a portion of step (IIa) occurs simultaneous with at least a portion of step (IIb), and at least a portion of step (IIc) occurs simultaneous with at least a portion of step (IIb).

A14: The process of A7 or A8, wherein step (II) comprises; step (IIa) then step (IIb) then step (IIc).

A15: The process of A7 or A8, wherein step (II) comprises: step (IIa) then step (IIb) and step (IIc) in either order and/or at least partially simultaneously.

A16: The process of A7 or A8, wherein step (II) comprises: at least a portion of step (IIa) occurs simultaneous with at least a portion of step (lib) and then step (IIc).

A17: The process of A7 or A8, wherein step (II) further comprises: (IId) removing at least a portion of water from the first methylation product mixture, and wherein step (IIa), step (IIb), step (IIc), and step (IId) independently occur in any order and/or simultaneously.

A18: The process of any of A1 to A7, wherein step (II) further comprises: (IId) removing at least a portion of water from the first methylation product mixture, and wherein step (IIa), step (IIb), and step (IId) independently occur in any order and/or simultaneously.

A19: The process of A18, wherein step (II) comprises: at least a portion of step (IIa) occurs simultaneous with at least a portion of step (IIb).

A20: The process of A18, wherein step (II) comprises: step (IIa) before or after step (IIb).

A21: The process of A18, wherein step (II) comprises: at least a portion of step (IId) occurs simultaneous with at least a portion of step (IIa).

A22: The process of A18, wherein step (II) comprises: step (IIa) before or after step (IId).

A23: The process of A18, wherein step (II) comprises: step (IIa) then step (IId) then step (IIb).

A24: The process of A18, wherein step (II) comprises: step (IId) then step (IIa) then step (IIb).

A25: The process of A18, wherein step (II) comprises: wherein step (II) comprises: step (IId) then step (IIa) then step (IIb), wherein at least a portion of step (IId) occurs simultaneous with at least a portion of step (IIa).

A26: The process of A18, wherein step (II) comprises: wherein step (II) comprises: step (IId) then step (IIa) then step (IIb), wherein at least a portion of step (IId) occurs simultaneous with at least a portion of step (IIa) and at least a portion of step (IIb) occurs simultaneous with at least a portion of step (IIa).

A27: The process of any of A1 to A26, wherein one or both of the first set of methylation conditions and the second set of methylation conditions comprise an absolute pressure, the same or different, in the range from 100 kPa to 8,500 kPa.

A28: The process of A27, wherein one or both of the first set of methylation conditions and the second set of methylation conditions comprise an absolute pressure, the same or different, in the range from 1000 kPa to 5000 kPa.

A29: The process of any of A1 to A28, wherein the first methylation catalyst and the second methylation catalyst have the same composition.

A30: The process of any of A1 to A29, wherein the first methylation catalyst and the second methylation catalyst both comprise a zeolite, the same or different, of the MWW framework type.

A31: The process of A30, wherein the zeolite of the MWW framework type is selected from MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, UCB-3, and mixtures of two or more thereof.

A32: The process of A30, wherein the zeolite of the MWW framework type is selected from MCM-22, MCM-49, MCM-56, and mixtures of two or more thereof.

A33: The process of any of A1 to A32, wherein at least one of the following is met:
(i) a ratio R(a/m)(1) as defined below is in a range from 2 to 10,

$$R ( a / m ) ( 1 ) = \frac{M ( tol ) ( 1 ) + 2 \cdot M ( bz ) ( 1 )}{M ( methanol ) ( 1 ) + 2 \cdot M ( DME ) ( 1 )}$$

where M(tol)(1) and M(bz)(1) are moles of toluene and benzene in the first aromatic hydrocarbon feed, respectively, and M(methanol)(1) and M(DME)(2) are moles of methanol and dimethyl ether in the first methylating agent feed; and
(ii) a ratio R(a/m)(2) as defined below is in a range from 2 to 10,

$$R ( a / m ) ( 2 ) = \frac{M ( tol ) ( 2 ) + 2 \cdot M ( bz ) ( 2 )}{M ( methanol ) ( 2 ) + 2 \cdot M ( DME ) ( 2 )}$$

where M(tol)(2) and M(bz)(2) are moles of toluene and benzene in the mixture feed, respectively, and M(methanol)(2) and M(DME)(2) are moles of methanol and dimethyl ether in the mixture feed, respectively.

A34: The process of A33, wherein at least one of the following is true: (i) 6≤R(a/m)(1)≤10; and (ii) 6≤R(a/m)(2)≤10.

A35: The process of any of A1 to A34, wherein the first fixed bed and the second fixed bed are contained in a one vessel.

A36: The process of any of A1 to A34, wherein the first fixed bed is contained in a first vessel, and wherein the second fixed bed is contained in a second vessel separate from the first vessel in fluid communication with the first vessel.

B1: A process for producing p-xylene, the process comprising: (I) contacting an aromatic hydrocarbon feed with a first methylating agent feed in the presence of a first methylation catalyst in a first fixed bed under a first set of methylation reaction conditions to produce a first methylation product mixture comprising p-xylene exiting the first fixed bed, wherein the aromatic hydrocarbon feed comprises benzene and/or toluene, wherein the first methylating agent feed comprises methanol and/or dimethyl ether, and wherein the first methylation product mixture when exiting the first fixed bed has a first temperature of T1; (II) producing a mixture feed having a second temperature of T2 via steps comprising: (IIa) reducing a temperature of the first methylation product mixture, (IIb) injecting a second methylating agent feed having a fourth temperature T4 into the first methylation product mixture, and (IIc) removing at least a portion of water from the first methylation product mixture, wherein the second methylating agent feed comprises methanol and/or dimethyl ether, and step (IIa), step (IIb), and step (IIc) independently occur in any order and/or simultaneously; and (III) contacting the mixture feed with a second methylation catalyst in a second fixed bed under a second set of methylation reaction conditions to produce a second methylation product mixture comprising p-xylene exiting the second fixed bed, wherein the second methylation product mixture when exiting the second fixed bed has a third temperature of T3.

B2: The process of B1, wherein 25° C.≤T1−T2≤200° C.

B3: The process of B1 or B2, wherein at least one of the following is met: 200° C.≤T1≤500° C.; and 200° C.≤T3≤500° C.

B4: The process of any of B1 to B3, wherein at least one of the following is met: 250° C.≤T1≤400° C.; and 250° C.≤T3≤400° C.

B5: The process of any of B1 to B4, wherein 25° C.≤T1−T4≤300° C.

B6: The process of any of B1 to B5, wherein step (IIa) comprises: transferring at least a portion of an amount of heat from the first methylation product mixture by using a heat exchanger.

B7: The process of any of B1 to B6, wherein step (II) comprises: at least a portion of step (IIa) occurs simultaneous with at least a portion of step (IIb).

B8: The process of any of B1 to B6, wherein step (II) comprises: step (IIa) before or after step (IIb).

B9: The process of any of B1 to B6, wherein step (II) comprises: at least a portion of step (IIc) occurs simultaneous with at least a portion of step (IIa).

B10: The process of any of B1 to B6, wherein step (II) comprises: step (IIa) before or after step (IIc).

B11: The process of any of B1 to B6, wherein step (II) comprises: step (IIa) then step (IIb) then step (IIc).

B12: The process of any of B1 to B6, wherein step (II) comprises: step (IIa) then step (IIc) then step (IIb).

B13: The process of any of B1 to B6, wherein step (II) comprises: wherein step (II) comprises: step (IIc) then step (IIa) then step (IIb), wherein at least a portion of step (IIc) occurs simultaneous with at least a portion of step (IIa).

B14. The process of any of B1 to B6, wherein step (II) comprises: wherein step (II) comprises: step (IIc) then step (IIa) then step (IIb), wherein at least a portion of step (IIc) occurs simultaneous with at least a portion of step (IIa) and at least a portion of step (IIb) occurs simultaneous with at least a portion of step (IIa).

B15: The process of any of B1 to B14, wherein one or both of the first set of methylation conditions and the second set of methylation conditions comprise an absolute pressure, the same or different, in the range from 100 kPa to 8,500 kPa.

B16: The process of B15, wherein one or both of the first set of methylation conditions and the second set of methylation conditions comprise an absolute pressure, the same or different, in the range from 1000 kPa to 5000 kPa.

B17: The process of any of B1 to B16, wherein the first methylation catalyst and the second methylation catalyst have the same composition.

B18: The process of any of B1 to B17, wherein the first methylation catalyst and the second methylation catalyst both comprise a zeolite, the same or different, of the MWW framework type.

B19: The process of B18, wherein the zeolite of the MWW framework type is selected from MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, UCB-3, and mixtures of two or more thereof.

B20: The process of B18, wherein the zeolite of the MWW framework type is selected from MCM-22, MCM-49, MCM-56, and mixtures of two or more thereof.

B21: The process of any of B1 to B20, wherein at least one of the following is met:
(i) a ratio R(a/m)(1) as defined below is in a range from 2 to 10,

$$R ( a / m ) ( 1 ) = \frac{M ( tol ) ( 1 ) + 2 \cdot M ( bz ) ( 1 )}{M ( methanol ) ( 1 ) + 2 \cdot M ( DME ) ( 1 )}$$

where M(tol)(1) and M(bz)(1) are moles of toluene and benzene in the first aromatic hydrocarbon feed, respectively, and M(methanol)(l) and M(DME)(2) are moles of methanol and dimethyl ether in the first methylating agent feed; and
(ii) a ratio R(a/m)(2) as defined below is in a range from 2 to 10,

$$R ( a / m ) ( 2 ) = \frac{M ( tol ) ( 2 ) + 2 \cdot M ( bz ) ( 2 )}{M ( methanol ) ( 2 ) + 2 \cdot M ( DME ) ( 2 )}$$

where M(tol)(2) and M(bz)(2) are moles of toluene and benzene in the mixture feed, respectively, and M(methanol)(2) and M(DME)(2) are moles of methanol and dimethyl ether in the mixture feed, respectively.

B22: The process of B21, wherein at least one of the following is true: (i) 6≤R(a/m)(1)≤10; and (ii) 6≤R(a/m)(2)≤10.

B23: The process of any of B1 to B22, wherein the first fixed bed and the second fixed bed are contained in a one vessel.

B24: The process of any of B1 to B22, wherein the first fixed bed is contained in a first vessel, and wherein the second fixed bed is contained in a second vessel separate from the first vessel in fluid communication with the first vessel.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of this disclosure, additionally, they do not exclude impurities and variances normally associated with the elements and materials used.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and/or testing procedures to the extent they are not inconsistent with this text. As is apparent from the foregoing general description and the specific embodiments, while forms of this disclosure have been illustrated and described, various modifications can be made without departing from the spirit and scope of this disclosure. Accordingly, it is not intended that this disclosure be limited thereby. Likewise, the term “comprising” is considered synonymous with the term “including” for purposes of United States law. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

While this disclosure has been described with respect to a number of embodiments and examples, those skilled in the art, having benefit of this disclosure, will appreciate that other embodiments can be devised which do not depart from the scope and spirit of this disclosure.

1. A process for producing p-xylene, the process comprising:
(I) contacting an aromatic hydrocarbon feed with a first methylating agent feed in the presence of a first methylation catalyst in a first fixed bed under a first set of methylation reaction conditions to produce a first methylation product mixture comprising p-xylene exiting the first fixed bed, wherein the aromatic hydrocarbon feed comprises benzene and/or toluene, wherein the first methylating agent feed comprises methanol and/or dimethyl ether, and wherein the first methylation product mixture when exiting the first fixed bed has a first temperature of T1;
(II) producing a mixture feed having a second temperature of T2 via steps comprising:
(IIa) reducing a temperature of the first methylation product mixture and
(IIb) injecting a second methylating agent feed having a fourth temperature T4 into the first methylation product mixture, wherein step (IIa) occurs before, after, and/or during step (IIb), and wherein the second methylating agent feed comprises methanol and/or dimethyl ether; and
(III) contacting the mixture feed with a second methylation catalyst in a second fixed bed under a second set of methylation reaction conditions to produce a second methylation product mixture comprising p-xylene exiting the second fixed bed, wherein the second methylation product mixture when exiting the second fixed bed has a third temperature of T3.

2. The process of claim 1, wherein 25° C.≤T1−T2≤200° C.

3. The process of claim 1, wherein at least one of the following is met:
200° C.≤T1≤500° C.; and
200° C.≤T3≤500° C.

4. The process of claim 1, wherein at least one of the following is met:
250° C.≤T1≤400° C.; and
250° C.≤T3≤400° C.

5. The process of claim 1, wherein 25° C.≤T1−T4≤300° C.

6. The process of claim 1, wherein step (IIa) comprises:
transferring at least a portion of an amount of heat from the first methylation product mixture by using a heat exchanger.

7. The process of claim 1, wherein step (II) further comprises:
(IIc) injecting a second aromatic hydrocarbon feed having a fifth temperature T5 into the first methylation product mixture, wherein the second aromatic hydrocarbon feed comprises benzene and/or toluene, and wherein step (IIa), step (IIb), and step (IIc) independently occur in any order and/or simultaneously.

8. The process of claim 7, wherein 25° C.≤T1−T5≤300° C., and wherein the mixture feed comprises the first methylation product mixture, the second methylating agent feed, and the second aromatic hydrocarbon feed.

9. The process of claim 1, wherein step (II) further comprises:
(IId) removing at least a portion of water from the first methylation product mixture, and wherein step (IIa), step (IIb), step (IIc), and step (IId) independently occur in any order and/or simultaneously.

10. The process of claim 1, wherein one or both of the first set of methylation conditions and the second set of methylation conditions comprise an absolute pressure, the same or different, in the range from 100 kPa to 8,500 kPa.

11. The process of claim 10, wherein one or both of the first set of methylation conditions and the second set of methylation conditions comprise an absolute pressure, the same or different, in the range from 1000 kPa to 5000 kPa.

12. The process of claim 1, wherein the first methylation catalyst and the second methylation catalyst have the same composition.

13. The process of claim 1, wherein the first methylation catalyst and the second methylation catalyst both comprise a zeolite, the same or different, of the MWW framework type.

14. The process of claim 13, wherein the zeolite of the MWW framework type is selected from MCM-22, PSH-3, SSZ-25, ERB-1, ITQ-1, ITQ-2, MCM-36, MCM-49, MCM-56, EMM-10, EMM-12, EMM-13, UZM-8, UZM-8HS, UZM-37, UCB-3, and mixtures of two or more thereof.

15. The process of claim 13, wherein the zeolite of the MWW framework type is selected from MCM-22, MCM-49, MCM-56, and mixtures of two or more thereof.

16. The process of claim 1, wherein at least one of the following is met:
(i) a ratio R(a/mxl) as defined below is in a range from 2 to 10, “R” (“a”/“m”)“(1)=” “M” (“tol”)“(1)+2·M(bz)(1)”/“M” (“methanol”)“(1)+2·M(DMEX1)” where M(tol)(1) and M(bz)(1) are moles of toluene and benzene in the first aromatic hydrocarbon feed, respectively, and M(methanol)(1) and M(DME)(2) are moles of methanol and dimethyl ether in the first methylating agent feed; and
(ii) a ratio R(a/m)(2) as defined below is in a range from 2 to 10, “R” (“a”/“m”)“(2)=” “M” (“tol”)“(2)+2·M(bz)(2)”/“M” (“methanol”)“(2)+2·M(DMEx2)” where M(tol)(2) and M(bz)(2) are moles of toluene and benzene in the mixture feed, respectively, and M(methanol)(2) and M(DME)(2) are moles of methanol and dimethyl ether in the mixture feed, respectively.

17. The process of claim 16, wherein at least one of the following is true:
(i) 6≤R(a/m)(1)≤10; and
(ii) 6≤R(a/m)(2)≤10.

18. The process of claim 1, wherein the first fixed bed and the second fixed bed are contained in a one vessel.

19. The process of claim 1, wherein the first fixed bed is contained in a first vessel, and wherein the second fixed bed is contained in a second vessel separate from the first vessel in fluid communication with the first vessel.

20. A process for producing p-xylene, the process comprising:
(I) contacting an aromatic hydrocarbon feed with a first methylating agent feed in the presence of a first methylation catalyst in a first fixed bed under a first set of methylation reaction conditions to produce a first methylation product mixture comprising p-xylene exiting the first fixed bed, wherein the aromatic hydrocarbon feed comprises benzene and/or toluene, wherein the first methylating agent feed comprises methanol and/or dimethyl ether, and wherein the first methylation product mixture when exiting the first fixed bed has a first temperature of T1;
(II) producing a mixture feed having a second temperature of T2 via steps comprising:
(IIa) reducing a temperature of the first methylation product mixture,
(IIb) injecting a second methylating agent feed having a fourth temperature T4 into the first methylation product mixture, and
(IIc) removing at least a portion of water from the first methylation product mixture, wherein the second methylating agent feed comprises methanol and/or dimethyl ether, and step (IIa), step (IIb), and step (IIc) independently occur in any order and/or simultaneously; and
(III) contacting the mixture feed with a second methylation catalyst in a second fixed bed under a second set of methylation reaction conditions to produce a second methylation product mixture comprising p-xylene exiting the second fixed bed, wherein the second methylation product mixture when exiting the second fixed bed has a third temperature of T3.
